(19) United States
(12) Patent Application Publication
(10) Pub. No.: US 2011/0112564 A1
(43) Pub. Date: May 12, 2011

(54) Device And Method For Crossing Occlusions

(76) Inventor: Yehuda G. Wolf, Mevaseret Zion (IL)

(21) Appl. No.: 12/993,326

(22) PCT Filed: May 21, 2009
(86) PCT No.: PCT/IL09/00504

§ 371 Date: Jan. 18, 2011

Related U.S. Application Data
(60) Provisional application No. 61/054,815, filed on May 21, 2008.
(60) Provisional application No. 61/199,040, filed on Nov. 13, 2008.

(51) Int. Cl. A61B 17/22

(52) U.S. Cl. 606/159

(57) ABSTRACT

A catheter (1) adapted to cross occlusions in vessels of a patient. The catheter is having a proximal end reversibly insertable adjacent to the occlusion and a distal opposite end located outside the patient. When the pusher (10) in the OPEN configuration, it provides a preset void volume barriered by means of the pusher, the proximal end of the catheter and the inner surface of the vessel. The vacuum generating mechanism applies suction on the occlusion and on the inner surface of the vessel through the void volume, to collapse and fix the tissues around the pusher and to stabilize the pusher of the catheter so that the occlusion can be penetrated with a guide-wire (13).

### FIELD OF THE INVENTION

The present invention generally pertains to a device and method for crossing occlusions in vessels of a patient with a guide-wire facilitated in a catheter and more specifically for treating chronic total (arterial) occlusion (CTO).

### BACKGROUND OF THE INVENTION

The present invention relates to Chronic Total Occlusions (CTO's) and especially in arteries. Chronic total occlusions in arteries, both in coronary and peripheral arteries (non-coronary), constitute the most common reason for failure of endovascular (percutaneous, trans-catheter) treatment and referral to open surgery. These arterial lesions constitute the biggest and most important challenge in endovascular therapy. A prerequisite to endovascular treatment of these lesions is the crossing of the occlusions with a guide-wire (from patent lumen on one side to the other) on which therapeutic interventions such as balloon dilation, stent placement and other interventions can be performed. In order to achieve this crossing consistently, reliably and safely, new devices and instrumentation are needed.

The most common cause of arterial occlusive disease is atherosclerosis. Atherosclerosis is a chronic, progressive disease of the arteries in which “plaques” made up of cholesterol deposits, calcium, and abnormal cells develop on the inner lining of the arteries.

Chronic total occlusions exist in both coronary and peripheral arteries, but have been histologically characterized mostly in the coronary arteries. It has been shown that their composition is irregular and changes over time. Specifically, the plaque is not homogenous and contains regions of different composition and structure with multiple neovascular channels.

Outside the lesion and within the outer layer of the arterial wall (the adventitia), the subintimal plane is a consistent potential space of loosely adherent layers. In an effort to avoid the complications associated with open surgical operations, wire-based devices for endovascular treatment of CTO have been introduced. Traditionally, it has been accepted that staying within the lumen and the plaque, rather than outside it, is preferred, so that when the guide-wire exits the occlusion, it is within the arterial lumen beyond it. However, traversing the plaque, which may be hard and calcified, is often difficult even with the use special high-end guide-wires with very stiff tips. To increase the success rate, several devices have been or are being developed. These include among others the Frontrunner (Cordis, J&J) which is based on blunt microdissection, The Crosser (Flowcardia Sunnyvale Calif.) a guide-wire with vibrational energy, the Safe-cross (Intraluminal Therapeutics, Carlsbad Calif.) wire with radiofrequency energy and OCT guidance, the Excimer laser catheter (Spectranetics, Colorado Springs Colo.), CTOS oscillating guide-wire (ReVascular therapeutics Sunnyvale Calif.), CiTop guide-wire (Ovalum Medical Vision, Rehovot Israel) with steerable J-tip guide-wire control and the Niobe system for manipulating instrumentation within magnetic fields. In addition, pharmaco-therapy with local injection of collagenase (Bradley Strauss, Toronto) is being investigated.

### Subintimal Angioplasty

An important endovascular technique to overcome total occlusions is subintimal angioplasty, where a dissection plane is intentionally developed beside the occlusion in the subintimal plane (outside the natural arterial lumen and outside the atherosclerotic plaque, outside the intima and inside the adventitia). This plane is entered from the lumen proximal to the occlusion and the device (wire or catheter) within it is advanced to beyond the occlusion. At this point and preferably not too far away from the termination of the occlusion, the lumen has to be re-entered.

The appeal of the subintimal route is that it is always present, irrespective of the structure and composition of the atherosclerotic plaque. It may be difficult to enter this plane with a guide-wire. This requires puncturing the intima at some point proximal to the occlusion to reach this plane from the arterial lumen. Once inside this plane with a guide-wire, the wire can usually be advanced with a relatively low resistance. The most difficult and least controlled step in this procedure is the reentry. This last step is occasionally difficult and if the guide-wire does not re-enter the true lumen close to the occlusion the entire endovascular procedure may have to be abandoned. Once the guide-wire has entered the lumen beyond the occlusion,, the intervention proceeds as usual with balloon dilation and stent application, as required.

Three devices which have been designed to control the re-entry site are on the market. Two devices are similar and use a curved hollow needle, which is driven off center (sideways), to perforate the intima and enter the lumen from the subintimal plane at the desired location. Through this needle, a guide-wire is advanced to the distal lumen, and from this point on the procedure can proceed as usual.

One device is the Outback reentry catheter by Cordis J&J and the other is the Pioneer catheter by Medtronic. Both catheters have a large profile (Outback 6F, Pioneer 7F) which is a disadvantage, especially for use in smaller arteries. Both catheters require precise rotational orientation, so that the needle is driven into the true lumen and not outside the adventitia and the artery. The outback relies on fluoroscopy and a radio-opaque rotation marker, the Pioneer incorporates an IVUS (intravascular ultrasound) catheter for orientation. A third device is a reentry balloon catheter where a flat low profile balloon helps orient the wire towards the lumen (Bridgepoint medical, Minneapolis, Minn.)

Crossing of a CTO in practice: While techniques of crossing the occlusion within the plaque (i.e. intra-luminally) and subintimal angioplasty are presented above as two profoundly different procedures, they are in essence quite similar. In fact, it is occasionally difficult to judge along which plane the guide-wire has actually crossed the total occlusion, whether within the plaque or in the subintimal plane.

The interior of the occluding plaque is inhomogeneous in terms of composition and structure and even within the subintimal plane there may be varying resistance to guide-wire advancement as well. Therefore a means to enhance passage of a guide-wire, to the desired site of reentry, may be helpful.

It is therefore a long felt need to provide a device and method especially adapted to cross occlusions. The device will enable reentry of the guide-wire from the subintimal space into vessel and will also stabilize the tip of the device for advancement of a wire within the lesion.

### SUMMARY OF THE INVENTION

It is one object of the present invention to provide a catheter for crossing occlusions in vessels of a patient. The catheter has a proximal end reversibly insertable within the occlusion and a distal opposite end located outside the patient. The catheter is characterized by vacuum generating mechanism (VGM), lumen in fluid communication with the VGM, pusher and a guide-wire maneuverable within the lumen. The pusher and/or the guide-wire are adapted to at least partially cross the occlusion while an effective measure of vacuum, provided as at least one fluid pulse or other manner, towards the occlusion.

It is another object of the present invention to provide a catheter which comprises at least one open-bore lumen provided in parallel to the main longitudinal axis of the catheter, having a proximal end located at the proximal end of the catheter, and a distal end located outside the patient. The catheter also comprises at least one vacuum generating mechanism (VGM), being in a fluid communication with the lumen's proximal end. The VGM is adapted for generating an effective measure of vacuum and introducing the vacuum towards the proximal end of the catheter, via a portion or an entire length of the lumen. The catheter also comprises at least one movable pusher, adapted. to be reversibly reciprocated, by means of an actuating mechanism, along and around the main longitudinal axis of the catheter. The pusher is locatable between at least two configurations, a CLOSE configuration and an OPEN configuration, in the OPEN configuration, the proximal end of the pusher is intimately anchored within the occlusion. The catheter also comprises at least one guide-wire, accommodated within the lumen, reciprocally maneuvered along and around the main longitudinal axis of the lumen. The guide-wire is adapted for crossing the occlusion. Wherein the pusher, in the OPEN configuration, provides within the vessel a preset void volume barriered by means of the pusher, the proximal end of the catheter and the inner surface of the occlusion. Wherein the VGM applies suction within the occlusion. Further wherein the pusher is stabilized and fixed within the occlusion to enable the guide-wire to cross the occlusion and to anchor it in an effective manner.

It is another object of the present invention to provide the catheter as defined above, adapted to treat patients with CTO conditions.

It is another object of the present invention to provide the catheter as defined above, wherein the actuating mechanism is at least one pushing wire. The pushing wire mechanically connected to the pusher. The pushing wire is maneuvered along and around the main longitude axis of the lumen. The pushing wire is adapted to actuate of the reciprocate movement of the pusher.

It is another object of the present invention to provide the catheter as defined above, wherein the actuating mechanism is at least one pushing wire. The pushing wire mechanically connected to the pusher. The pushing wire is maneuvered along and around the lumen. The pushing wire adapted for control of the movement of the pusher.

It is another object of the present invention to provide the catheter as defined above, wherein the pushing wire is a non-flexible wire.

It is another object of the present invention to provide the catheter as defined above, wherein at least one of the lumen is a pusher-lumen adapted to accommodate the pushing wire.

It is another object of the present invention to provide the catheter as defined above, wherein at least one of the lumen is a suction lumen adapted for fluid communication with the VGM.

It is another object of the present invention to provide the catheter as defined above, wherein at least one of the lumen is a guide-wire-lumen adapted to accommodate the guide-wire.

It is another object of the present invention to provide the catheter as defined above, wherein the pusher is a solid cap having a tapered shape, adapted to facilitate advancing, penetrating, anchoring, piercing, crossing or otherwise actuating of the catheter within the occlusion.

It is another object of the present invention to provide the catheter as defined above, wherein at least a proximal portion of the pusher is a curled wire, characterized by a shape selected from a group consisting of helix, sphere, cone, cylinder, egg-like, and any combination thereof.

It is another object of the present invention to provide the catheter as defined above, the pusher is characterized by shape memory.

It is another object of the present invention to provide the catheter as defined above, wherein the pusher is the continuation of the pushing wire.

It is another object of the present invention to provide the catheter as defined above, wherein the pusher is mechanically integrated with the pushing wire.

It is another object of the present invention to provide the catheter as defined above, wherein the guide-wire is characterized by a flexible tip. The tip is located at the proximal end of the guide-wire.

It is another object of the present invention to provide the catheter as defined above, wherein the guide-wire is characterized by a stiff tip. The tip is located at the proximal end of the guide-wire.

It is another object of the present invention to provide the catheter as defined above, wherein the guide-wire is an elongated straight member.

It is another object of the present invention to provide the catheter as defined above, wherein the guide-wire is characterized by a bent tip. The tip is located at the proximal end of the guide-wire.

It is another object of the present invention to provide the catheter as defined above, wherein the guide-wire is characterized by a sharpened tip adapted for puncturetion of the occlusion and of the layers of the vessel.

It is still an object of the present invention to provide a method for crossing an occlusion in vessels of a patient. The method comprises steps selected inter alia from: Providing at least one catheter adapted to cross occlusions in vessels of a patient having a proximal end reversibly insertable within the occlusion and a distal opposite end located outside the patient. The catheter also comprises at least one open-bore lumen provided in parallel to the main longitudinal axis of the catheter, having a proximal end located at the proximal end of the catheter, and a distal end located outside the patient. The catheter also comprises at least one vacuum generating mechanism (VGM), being in a fluid communication with the lumen's proximal end. The VGM is adapted for generating an effective measure of vacuum and introducing the vacuum towards the proximal end of the catheter, via a portion or an entire length of the lumen. The catheter also comprises at least one movable pusher; adapted to be reversibly reciprocate, by means of an actuating mechanism, along and around the main longitudinal axis of the catheter. The pusher is locatable between at least two configurations, a CLOSE configuration and an OPEN configuration, in the OPEN configuration, the proximal end of the pusher is intimately anchored within the occlusion. The pusher, in the OPEN configuration, provides within the vessel a preset void volume barriered by means of the pusher, the proximal end of the catheter and the inner surface of the occlusion. The catheter also comprises at least one guide-wire, accommodated within the lumen, reciprocally maneuvered along and around the main longitudinal axis of the lumen. The guide-wire is adapted for crossing the occlusion; Crossing the occlusion with the guide-wire or otherwise inserting the catheter to a location adjacent to the occlusion; Bringing the pusher into the OPEN configuration by moving the pusher forward, or alternatively, by moving the catheter backward while keeping the pusher adjacent to the occlusion; Generating vacuum by the VGM and applying suction within the occlusion. In this position, the pusher is stabilized and fixed to the inner surface of the occlusion and/or to the inner surface of the vessel to enable the guide-wire to cross the occlusion; Advancing the guide-wire through the lumen into the occlusion while the suction is applied and crossing the occlusion with the guide-wire; Advancing the catheter through the occlusion; In case of subintimal passage, once the occlusion has been passed with the catheter, and the catheter is within the vessel wall in subintimal position, verifying the position of the true lumen of the vessel and performing reentry with the guide-wire. if this step was not successful, continuing to step (h); Moving the guide-wire to a direction distal to the occlusion. bringing pusher to the OPEN configuration. creating vacuum by the VGM and applying suction on the inner surface of the occlusion and/or on the inner surface of the vessel through the void volume. In this position, the pusher is stabilized and fixed to the inner surface of the occlusion and/or to the inner surface of the vessel. Puncturating the intima of the vessel. Advancing guide wire either straight or bent in the direction of the lumen in order to puncture the intima (which is fixed to the pusher by suction) of the vessel in, order to gain access to the lumen; Once the guide-wire crossed the occlusion, terminating the suction, bringing the pusher into a the CLOSE configuration, the catheter is removed and the procedure continues with the guide-wire. Alternatively, the catheter advances beyond the occlusion for injection and visualization before its removal; and, Repeating at least one of the previous steps if required.

It is still an object of the present invention to provide the method as defined above, further comprising steps of treating patient with CTO conditions.

It is still an object of the present invention to provide the method as defined above, wherein the step of providing the catheter comprises at least one pushing wire; the pushing wire mechanically connected to the pusher.

It is still an object of the present invention to provide the method as defined above, wherein the step of bringing the pusher into the OPEN configuration comprises actuating the reciprocate movement of the pusher by the maneuvering the pushing wire along and around the main longitude axis of the lumen.

It is still an object of the present invention to provide the method as defined above, wherein the pushing wire is a non-flexible wire.

It is still an object of the present invention to provide the method as defined above, wherein the steps of providing the catheter and bringing the pusher into the OPEN configuration, the lumen is a pusher-lumen; the pushing wire is accommodated within the pusher-lumen.

It is still an object of the present invention to provide the method as defined above, wherein the step of providing the catheter and generating vacuum by the VGM, the lumen is a suction lumen fluidly communicating with the VGM.

It is still an object of the present invention to provide the method as defined above, wherein the step of providing the catheter, the at least one of the lumen is a guide-wire-lumen; the guide-wire is accommodated within the guide-wire-lumen.

It is still an object of the present invention to provide, the method as defined above, wherein the step of providing the catheter, the pusher is a solid cap having a tapered shape; the cap is advancing, penetrating, anchoring, piercing, crossing or otherwise actuating the catheter within the occlusion.

It is still an object of the present invention to provide the method as defined above, wherein the step of providing the catheter, least a proximal portion of the pusher is a curled wire, characterized by a shape selected from a group consisting of helix, sphere, cone, cylinder, egg-like, and any combination thereof.

It is still an object of the present invention to provide the method as defined above, wherein the pusher is characterized by shape memory.

It is still an object of the present invention to provide the method as defined above, wherein the pusher is mechanically integrated with the pushing wire.

It is still an object of the present invention to provide the method as defined above, wherein the step of providing the catheter, the guide-wire is characterized by a flexible tip; the tip is located at the proximal end of the guide-wire.

It is still an object of the present invention to provide the method as defined above, wherein the step of providing the catheter, the guide-wire is characterized by a stiff tip; the tip is located at the proximal end of the guide-wire.

It is still an object of the present invention to provide the method as defined above, wherein the step of providing the catheter, the guide-wire is an elongated straight member.

It is still an object of the present invention to provide the method as defined above, wherein the step of providing the catheter, the guide-wire is characterized by a bent tip; the tip is located at the proximal end of the guide-wire.

It is still an object of the present invention to provide the method as defined above, wherein the step of providing the catheter, the guide-wire is characterized by a sharpened tip adapted for puncturetion of the occlusion and of the layers of the vessel.

It is still an object of the present invention to provide a catheter. The catheter also comprises at least one fluid pressure-pulse generator adapted to provide at least one fluid pulse with relatively steep potential difference between initiation and termination. The catheter also comprises at least one lumen. The lumen is in communication with the fluid pressure-pulse generator. Wherein the pressure generator is adapted to provide the at least one fluid pulse through the catheter to the occlusion such that the occlusion is crossed.

It is still an object of the present invention to provide a catheter adapted to treat patients with CTO conditions.

It is still an object of the present invention to provide a catheter additionally comprising a movable pusher attached to the proximal end of the catheter that comes into contact with the occlusion.

It is still an object of the present invention to provide a catheter adapted to reverse the generation of pulses, and thereby to act in a suction manner.

It is still an object of the present invention to provide a method for crossing an occlusion in vessels of a patient, comprising:
a. obtaining at least one catheter adapted to cross an occlusion in a hollow, tube-like passage. The catheter also comprises at least one fluid pressure-pulse generator adapted to provide at least one fluid pulse with relatively steep potential difference between initiation and termination. The catheter also comprises at least one lumen. The lumen is in communication with the fluid pressure-pulse generator;
b. attempting to cross the occlusion with a regular guide-wire;
c. bringing the tip of the hydraulic wire in contact with the occlusive lesion;
d. applying a primary series of hydraulic impacts; thereby enabling passage of a guide-wire through the the occlusion;
e. advancing the wire through the occlusion.

f. It is still an object of the present invention to provide a method as described above, additionally comprising steps of
a. delivering a secondary series of hydraulic impacts to create localized disruption of the occluding tissue in order to enable passage of the wire; and,
b. entering the lumen beyond the occluded segment.

### Vacuum Generating Mechanism (VGM):

It is according to one embodiment of the invention wherein the vacuum generating mechanism produces negative pressure (suction) and can be connected to the catheter. The negative pressure is controllable and a transparent chamber shows whether body fluid (e.g. blood) is removed. The mechanism may have a negative pressure chamber through which the distal end of the catheter may be passed. This chamber has an air-tight sealing around the entry and exit sites of the catheter so that suction may be applied to the catheter which lacks a special connection port. Alternatively, the vacuum generating mechanism may be connected to a suction port in the catheter by conventional means (e.g., luer lock).

It is according to one embodiment of the invention wherein the catheter has an outer diameter of e.g., about 0.035″ to 4F (0.89-1.33 mm) The term ‘about’ refers hereinafter to any value being ±25% of the defined measure.

It is according to another embodiment of the invention wherein the guide-wire has a diameter between e.g., about 0.008″ to about 0.018″.

It is according to another embodiment of the invention wherein the pusher is in a configuration of a curled wire.

It is according to yet another embodiment of the invention wherein the pusher is at least partially made of a shape memory alloy (e.g., Nitinol) with a front segment which when unconstrained, takes on a three dimensional form to occupy space and prevent collapse of tissue into its inner space. One possible shape among many is a helix with a diameter similar to or somewhat larger than that of the suction wire or catheter. Other shapes include a variety of patterns creating a surface contour of a sphere, a cone, a cylinder or an egg-like form. The tip (front end) of the thin preformed spacing wire is a sphere with a diameter which may be larger than of the catheter, or is tightly curled up so it does not have a sharp pointing tip, but rather an atraumatic one.

### BRIEF DESCRIPTION OF THE FIGURES

In order to better understand the invention and its implementation in a practice, a plurality of embodiments will now be described, be way of non-limiting example only, with reference to the accompanying drawings, in which

FIG. 1 illustrates a catheter with an external diameter of approximately 0.8-1.5 mm, which has one or more lumens along all or part of its length;

FIG. 2 illustrates another embodiment of the catheter from FIG. 1;

FIG. 3 illustrates the catheter as illustrated in FIG. 1 with a pusher. The pusher in this figure is a wire which has an unconstrained configuration of a helix;

FIG. 4 illustrates the catheter 1 as illustrated in FIG. 3 with a guide-wire having an angled tip;

FIG. 5 illustrates the catheter 1 as illustrated in FIG. 4 with a guide-wire having a straight tip;

FIG. 6 illustrates the catheter as illustrated in FIG. 1 with a pusher and straight guide-wire. The pusher in this figure is a solid cap having a tapered shape. The pusher in this figure is in a CLOSE configuration;

FIG. 7 illustrates the catheter as illustrated in FIG. 6 with a pusher and straight guide-wire. The pusher in this figure is in an OPEN configuration;

FIG. 8 illustrates the catheter as illustrated in FIG. 7 with a pusher a guide-wire. The guide-wire has a rounded tip;

FIG. 9 illustrates wire having an, external diameter preferably but not necessarily 0.0035, which preferably but not necessarily has a weighted tip. The tip is preferably straight as shown, or alternatively angled. The lumen ends in a lateral aperture at some distance form the tip;

FIG. 10 illustrates a wire having a lumen which ends in 3 apertures near the tip with channels into apertures pointing from tip backwards;

FIG. 11 illustrates a wire having a lumen within external wall with 3 apertures near the tip and breakable point near the back end to which a tube connectable to a tube connector (such as a luer lock) is attached. Once the back end with the tube is broken off, a rod with a widened tip may be inserted into the lumen of the wire from the back to make the wire useful for passing catheters or other endovascular gear over it;

FIG. 12 illustrates a wire having a lumen within external wall 2 that ends in 3 apertures near the tip and ends with a lateral aperture near the back end where a sealed connection (too small for a screw connection) may be made with an attached tube or the aperture is contained within a high-pressure chamber in the pressure generator;

FIG. 13 illustrates a wire having a lumen with expandable tip at resting position. The tip is attached with springs to the shaft of the wire. At the lower portion of the movable tip there are apertures connected to the central lumen below the tip and covered within the front end of the shaft of the wire;

FIG. 14 illustrates a wire having a lumen with expandable tip with in expanded position. The tip which is attached with springs to the shaft of the wire, is pushed forward and the apertures, which in this drawing point backwards but may point sideways, are exposed beyond the walls of the shaft;

FIG. 15 illustrates a wire having a lumen with movable element. In the wire, 3 apertures near the tip and one aperture some distance back. Movable element is within the wire, has an aperture and a rod running through the wire so that it can be manipulated from the back end. In the position shown, apertures near the tip are covered and aperture some distance back is open through aperture in movable element;

FIG. 16 illustrates the same wire as in FIG. 15 with movable element pulled back so that back aperture is occluded (not aligned with aperture in movable element) and front apertures are open;

FIG. 17 illustrates catheter with lumen. Catheter over the wire (e.g. 3F), with a special channel and lumen for a thin wire (e.g. 0.014) and a separate lumen around the channel of the wire and within the catheter, ending near the front end with 3 apertures, close to the tip and the exit of the wire. The back end of the lumen exits sideways and ends in a connectable tube while the wire goes along the catheter to the back end; and,

FIG. 18 illustrates same catheter as in FIG. 17 but with rapid exchange configuration and exit of wire at mid-shaft.

### DETAILED DESCRIPTION OF THE PREFERRED EMBODIMENTS

According to one embodiment of the invention, a catheter is provided. The catheter is adapted to cross occlusions in vessels of a patient. The catheter is having a proximal end reversibly insertable adjacent to the occlusion and a distal opposite end located outside the patient. When the pusher in the OPEN configuration, it provides a catheter which is adapted to cross occlusions and methods thereof.

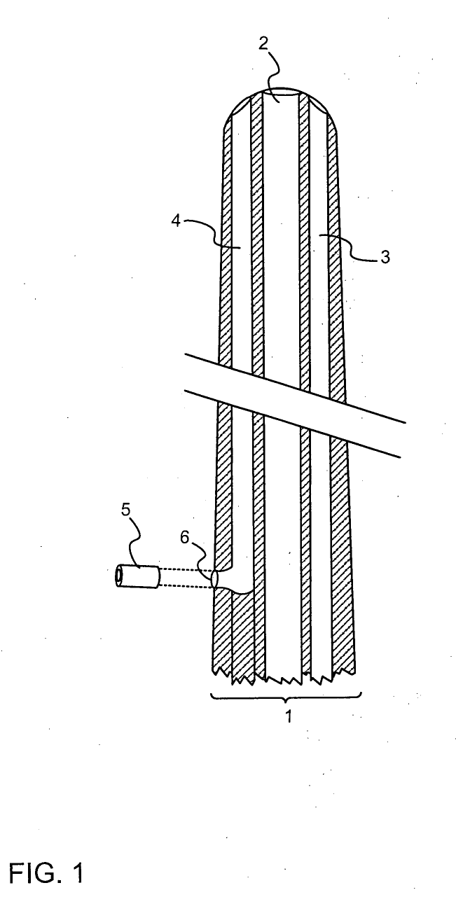

Reference is made now to FIG. 1 where the catheter 1 is schematically shown in an out-of-scale manner. The catheter has e.g., an external diameter of 0.8-1.5 mm, and has a pointed tip for advancing within occlusions. The catheter has a guide-wire lumen 2 which ends at the proximal end of the catheter. This lumen has a diameter of approximately 0.014″. This lumen may be central or eccentric in cross-section of the catheter, The lumen may be round or of a different shape (e.g. oval, cogwheel or other) and is designed to facilitate guiding wire (not shown). There is at least one pushing lumen 3 of another type, terminating beside the tip of the catheter. It is in the scope of the invention wherein the term ‘pushing lumen’ refers to a non-limiting manner to a conduit, pipe, hole, barrel, passageway, facilitated space, or other lumen adapted e.g., by means of size and shape to accommodate the pushing wire. The pushing lumen 3 is designed for accommodation of a pushing wire (not shown) or for the rods of a pusher (not shown). Suction lumen 4 extends along the proximal end of the catheter with an end hole beside end-holes of lumens 2 and 3. The distal end of this lumen is in a fluid communication with the vacuum generating mechanism by tubing and port 5. Alternatively it may end at a side-hole 6 near the distal end of 1 which can be inserted into a suction chamber within the vacuum generating mechanism.

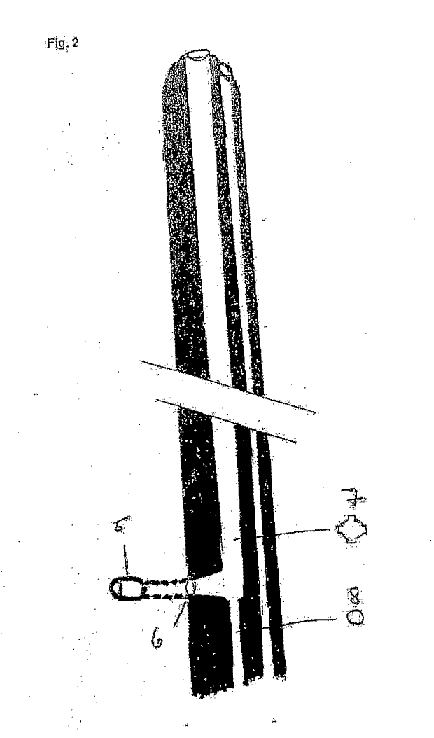

Reference is now made to FIG. 2 which illustrates another embodiment of the catheter schematically shown in an out-of-scale manner. It is in the scope of the invention wherein the term ‘guide-wire lumen’ refers to a non-limiting manner to a conduit, pipe, hole, barrel, passageway, facilitated space, or other lumen adapted e.g., by means of size and shape to accommodate the guide wire. In this embodiment all the components of the catheter are similar to components of the catheter from FIG. 1, and the only difference between the embodiments is that the guide-wire lumen 7 is combined with the suction lumen 8.

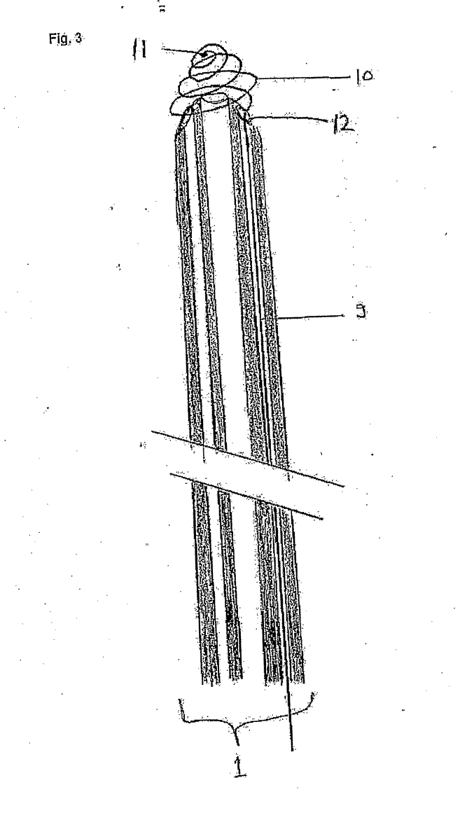

Reference is now made to FIG. 3 which illustrates the catheter 1 schematically shown in an out-of-scale manner, as illustrated in FIG. 1 with a pushing wire 9 in the pushing lumen 12. In this figure, pusher 11 is a wire which has an unconstrained configuration of a helix. The pusher 11 is the continuation of the pushing wire 9. The front tip of the pusher 11 has a sphere connected at the end which can serve to occlude the endhole of the lumen 12 if it is larger than the wire. Alternatively, pusher 11 may be tightly curled up so that it does not have a sharp pointed tip but an atraumatic one.

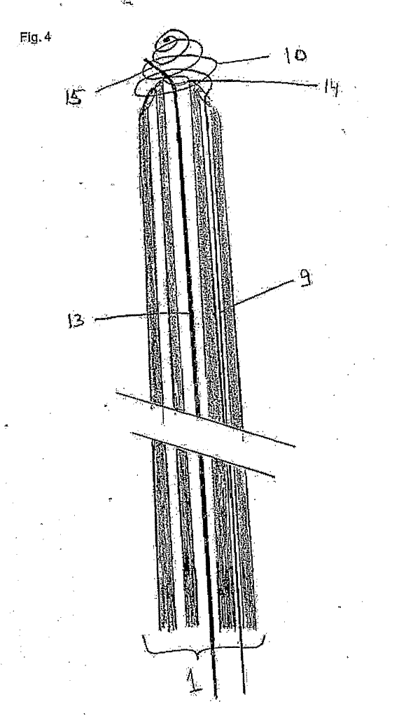

Reference is now made to FIG. 4 which illustrates the catheter 1 schematically shown in an out-of-scale manner as illustrated in FIG. 3 with a guide-wire 13. The guide-wire 13 is facilitated in the guide-wire lumen 14. The proximal end of the guide-wire 13 has an angled tip 15 and once outside the guide-wire lumen 14 it extends from the sideways of the pusher 10.

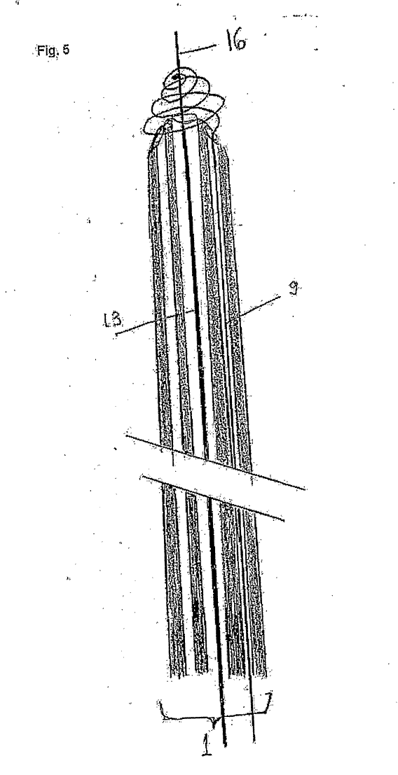

Reference is now made to FIG. 5 which illustrates the catheter 1 schematically shown in an out-of-scale manner, as illustrated in FIG. 4 with a guide-wire 13 that has straight tip 16. The straight tip 16 is extending through the center of the pusher 10.

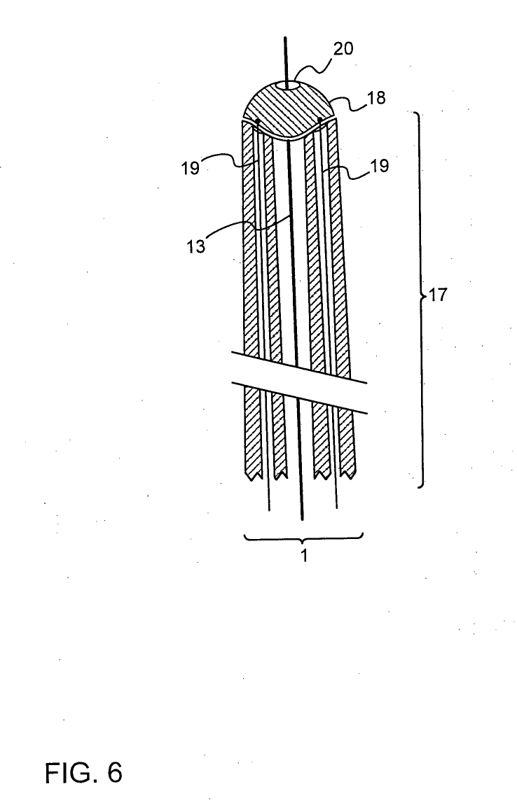

Reference is now made to FIG. 6 which illustrates another embodiment the catheter 1 schematically shown in an out-of-scale manner. In this embodiment the catheter 1 is the same as illustrated in FIG. 1. In this figure pusher 18 is mechanically connected to pushing wires 19 and is in a CLOSE configuration. The pusher 18 is a solid cap having a tapered shape to facilitate advancement of the catheter 1 within the occlusion. A guide-wire 13 is facilitated in the guide-wire lumen 14 and extending through the center of the pusher 18. The suction lumen is not illustrated in this figure but it exists either separately or in conjunction with one or more of the other lumens and terminates at the front surface of the body of the catheter.

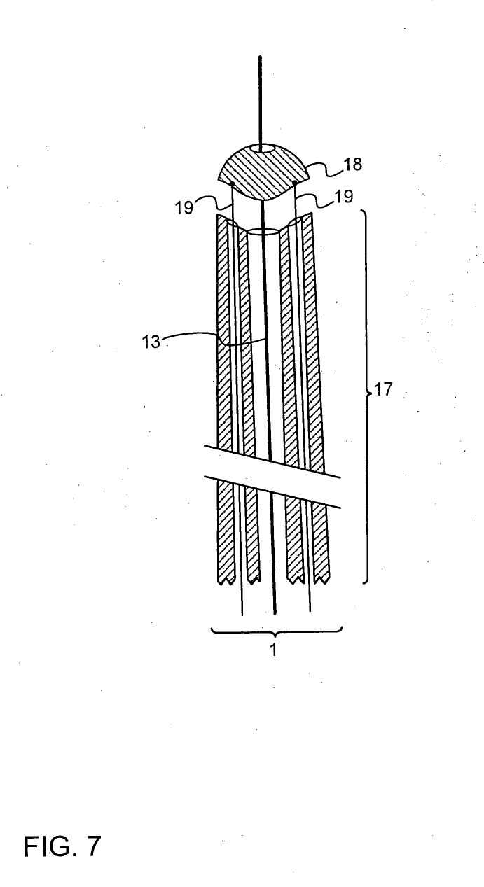

Reference is now made to FIG. 7 which illustrates the catheter 1 schematically shown in an out-of-scale manner, as illustrated in FIG. 6. In this figure all the components are the, same, but the pusher 18 is in an OPEN configuration.

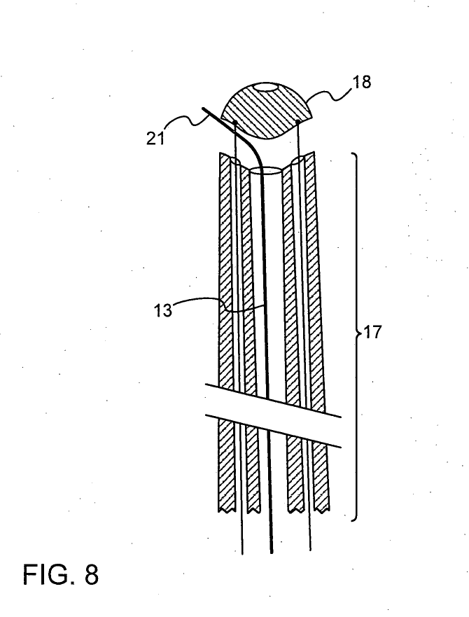

Reference is now made to FIG. 8 which illustrates the catheter 1 as illustrated in FIG. 7 schematically shown in an out-of-scale manner. In this figure all the components are the same, but the guide-wire has a rounded tip 21. The tip 21 is not extending trough the pusher 18, but by-pass it from the side.

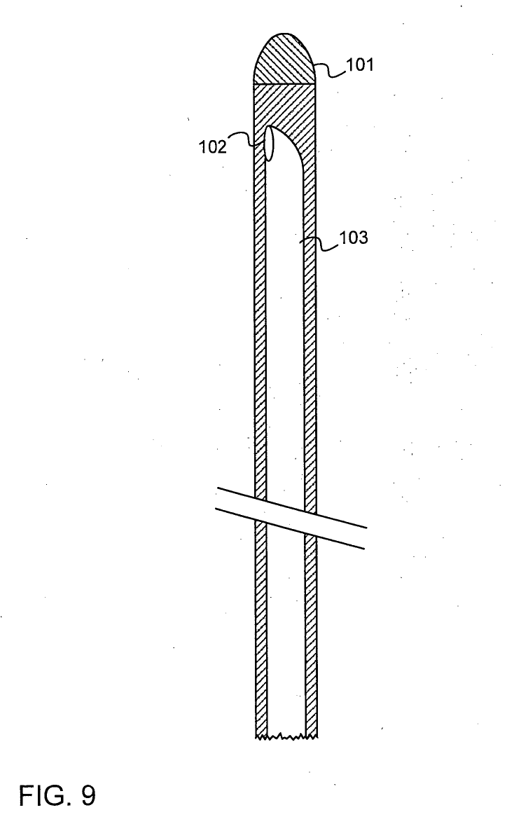

Reference is made now to FIG. 9 illustrating catheter 103 schematically shown in an out-of-scale manner having an external diameter preferably but not necessarily 0.0035, which preferably but not necessarily has a weighted tip 101. The tip is preferably straight as shown, or alternatively angled. The lumen ends in a lateral aperture 102 at some distance form the tip.

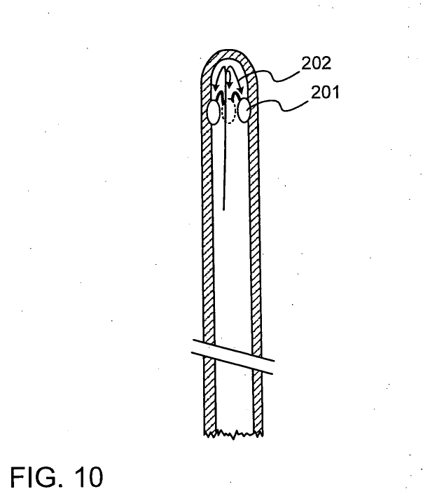

Reference is made now to FIG. 10 illustrating wire with lumen schematically shown in an out-of-scale manner, which ends in 3 apertures near the tip 201 with channels into apertures pointing from tip backwards 202.

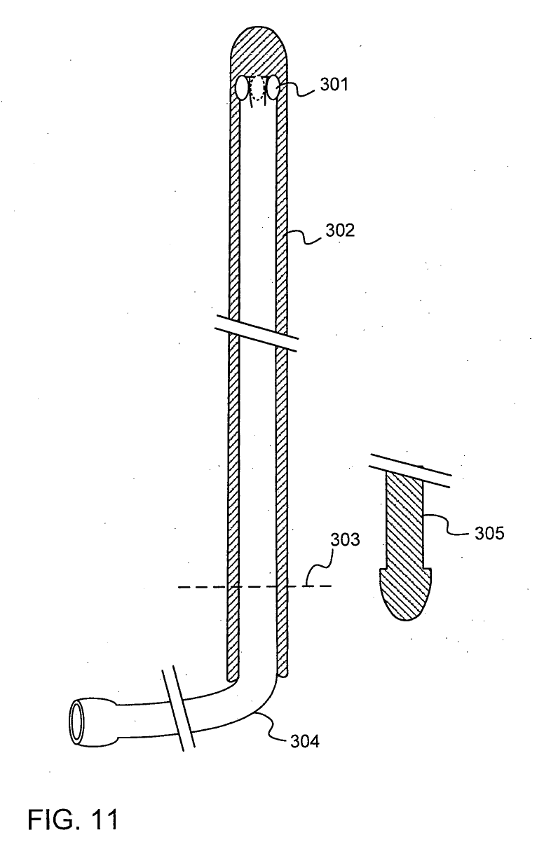

Reference is made now to FIG. 11 illustrating wire with lumen schematically shown in an out-of-scale manner, within external wall 302 with 3 apertures near the tip 301 and breakable point near the back end 303 to which a tube connectable to a tube connector (such as a luer lock) is attached 304. Once the back end with the tube is broken off at 303, a rod with a widened tip 305 may be inserted into the lumen of the wire from the back to make the wire useful for passing catheters or other endovascular gear over it.

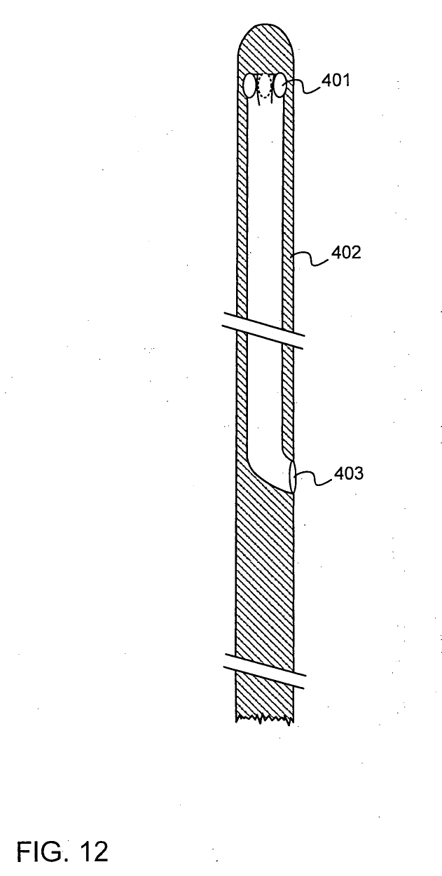

Reference is made now to FIG. 12 illustrating wire with lumen schematically shown in an out-of-scale manner, within external wall 2 that ends in 3 apertures near the tip 401 and ends with a lateral aperture near the back end 403 where a sealed connection (such as with a screw) may be made with an attached tube or the aperture is contained within a high-pressure chamber in the pressure generator.

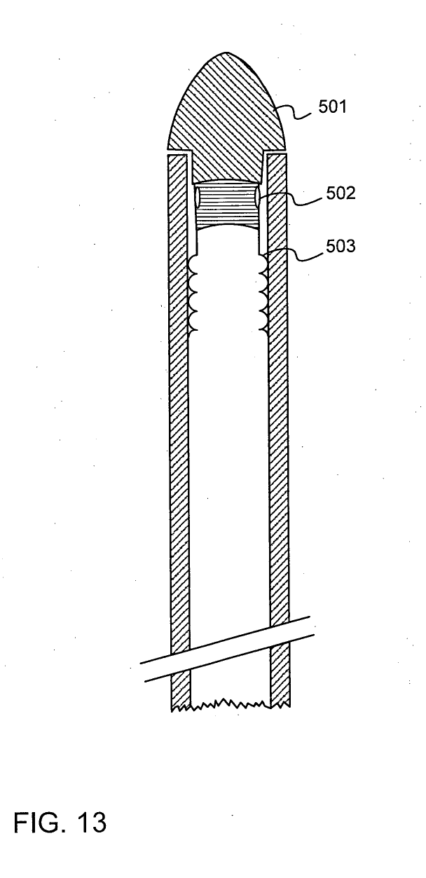

Reference is made now to FIG. 13 illustrating wire with lumen schematically shown in an out-of-scale manner, with expandable tip at resting position. The tip 501 is attached with springs 503 to the shaft of the wire. At the lower portion of the movable tip there are apertures connected to the central lumen below the tip and covered within the front end of the shaft of the wire.

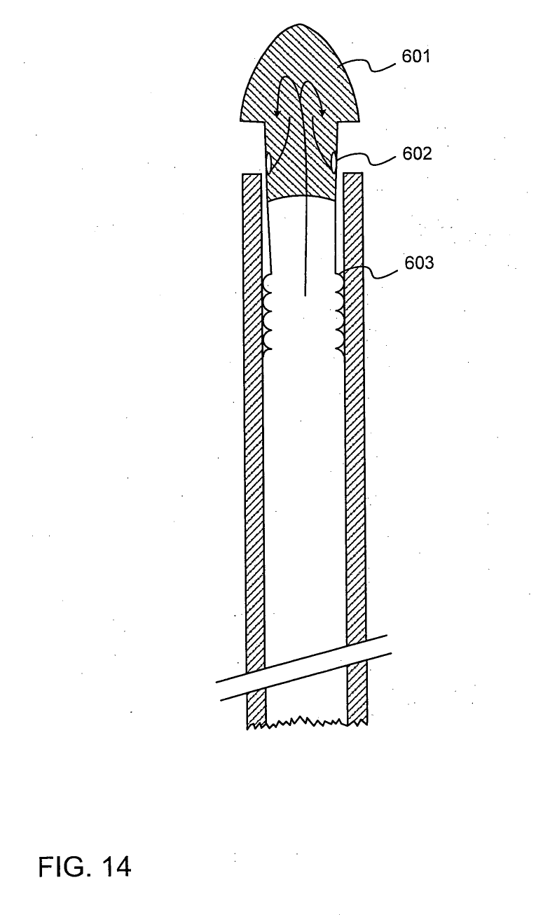

Reference is made now to FIG. 14 illustrating wire with lumen schematically shown in an out-of-scale manner, with expandable tip with in expanded position. The tip 601 which is attached with springs 603 to the shaft of the wire, is pushed forward and the apertures 602, which in this drawing point backwards but may point sideways, are exposed beyond the walls of the shaft.

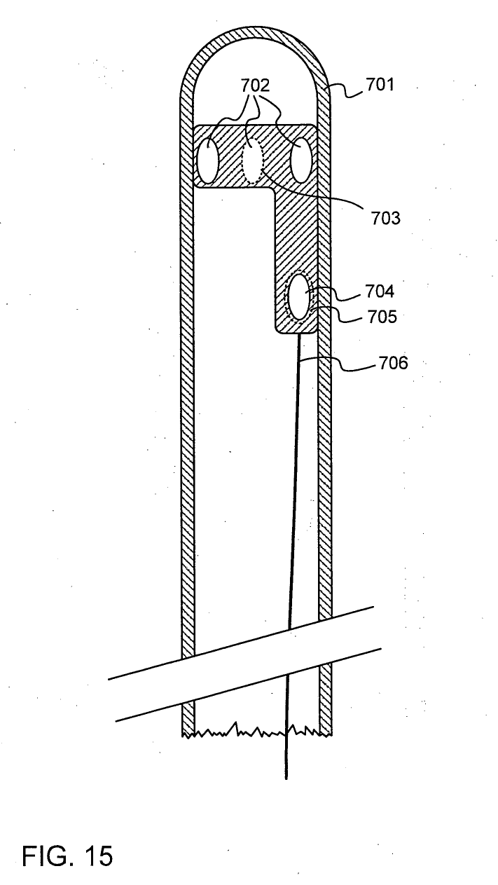

Reference is made now to FIG. 15 illustrating wire, with lumen 701 schematically shown in an out-of. scale manner, with movable element 703. In the wire, 3 apertures near the tip 702 and one aperture some distance back 704. Movable element is within the wire, has an aperture 705 and a rod running through the wire 706 so that it can be manipulated from the back end. In the position shown, apertures near the tip 702 are covered and aperture some distance back 704 is open through aperture 705 in movable element.

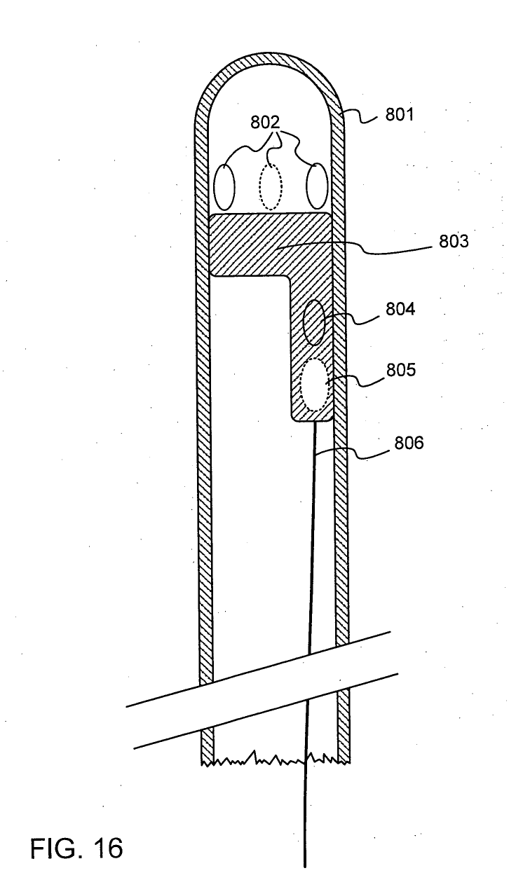

Reference is made now to FIG. 16 illustrating the same wire schematically shown in an out-of-scale manner, as in FIG. 15 with movable element pulled back so that back aperture 804 is occluded (not aligned with aperture 805 in movable element) and front apertures 802 are open.

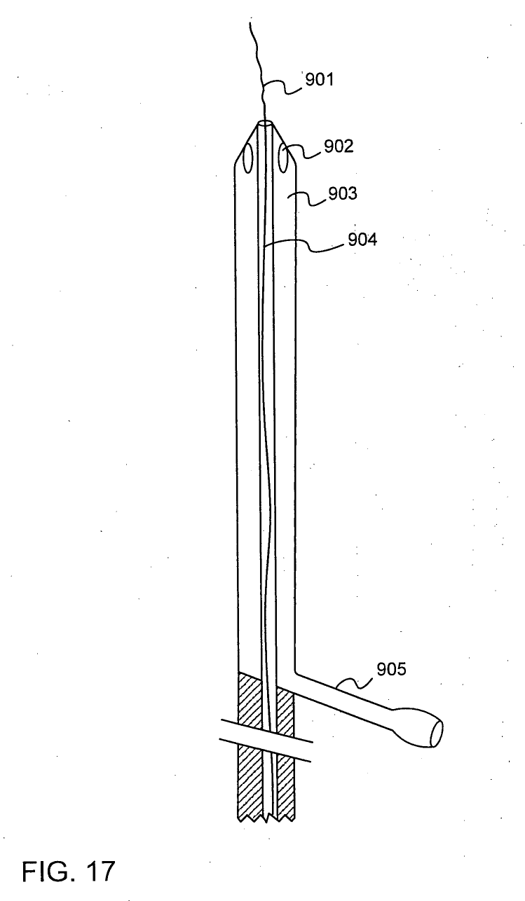

Reference is made now to FIG. 17 illustrating catheter with lumen schematically shown in an out-of-scale manner. Catheter over the wire (e.g. 3F), with a special channel and lumen for a thin wire (e.g. 0.014) 904 and a separate lumen around the channel of the wire and within the catheter 903, ending near the front end with 3 apertures 902, close to the tip and the exit of the wire 901. The back end of the lumen exits sideways and ends in a connectable tube 905 while the wire goes along the catheter to the back end.

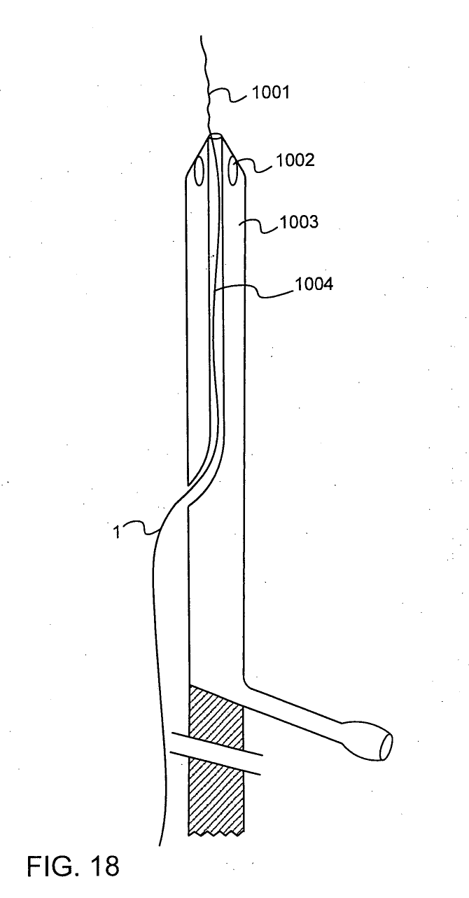

Reference is made now to FIG. 18 illustrating same catheter schematically shown in an out-of-scale manner, as in FIG. 17 but with rapid exchange configuration and. exit of wire 1001 at mid-shaft.

1. A catheter for crossing occlusions in vessels of a patient; said catheter having a proximal end reversibly insertable within said occlusion and a distal opposite end located outside the patient; said catheter is characterized by: vacuum generating mechanism (VGM); lumen in fluid communication with said VGM; pusher; guide-wire maneuverable within said lumen; said pusher and/or said guide-wire are adapted to at least partially cross said occlusion while an effective measure of vacuum, provided as at least one fluid pulse or other manner, towards said occlusion.

2. The catheter according to claim 1, comprising:
a. at least one open-bore lumen provided in parallel to the main longitudinal axis of said catheter, having a proximal end located at said proximal end of said catheter, and a distal end located outside said patient;
b. at least one vacuum generating mechanism (VGM), being in a fluid communication with said lumen's proximal end; said VGM is adapted for generating an effective measure of vacuum and introducing the vacuum towards said proximal end of said catheter, via a portion or an entire length of said lumen;
c. at least one movable pusher; adapted to be reversibly reciprocate, by means of an actuating mechanism, along and around said main longitudinal axis of said catheter; said pusher is locatable between at least two configurations, a CLOSE configuration and an OPEN configuration, in said OPEN configuration, the proximal end of said pusher is intimately anchored within said occlusion; and,
d. at least one guide-wire, accommodated within said lumen, reciprocally maneuvered along and around said main longitudinal axis of said lumen; said guide-wire is adapted for crossing said occlusion;
wherein said pusher, in said OPEN configuration, provides within said vessel a preset void volume barriered by means of said pusher, said proximal end of said catheter and the inner surface of said occlusion; wherein said VGM applies suction within said occlusion; further wherein said pusher is stabilized and fixed within said occlusion to enable said guide-wire to cross said occlusion and to anchor it in an effective manner.

3. The catheter according to claim 2, adapted to treat patients with CTO conditions.

4. The catheter according to claim 2, wherein said actuating mechanism is at least one pushing wire; said pushing wire mechanically connected to said pusher; said pushing wire is maneuvered along and around the main longitude axis of said lumen; said pushing wire is adapted to actuate of said reciprocate movement of said pusher.

5. (canceled)

6. The catheter according to claim 4, wherein at least one of said lumen is selected from a group consisting of (a) a pusher-lumen adapted to accommodate said pushing wire; (b) a suction lumen adapted for fluid communication with said VGM; (c) a guide-wire-lumen adapted to accommodate said guide-wire, or any combination thereof.

7. (canceled)

8. (canceled)

9. The catheter according to claim 2, wherein said pusher is a solid cap having a tapered shape, adapted to facilitate advancing, penetrating, anchoring, piercing, crossing or otherwise actuating of said catheter within said occlusion: further wherein said pusher is made of materials having shape memory properties.

10. The catheter according to claim 2, wherein at least a proximal portion of said pusher is a curled wire, characterized by a shape selected from a group consisting of helix, sphere, cone, cylinder, egg-like, and any combination thereof.

11. (canceled)

12. (canceled)

13. The catheter according to claim 2, wherein said guide-wire is characterized by at least one selected from a group consisting of (a) a flexible tip; said tip is located at the proximal end of said guide-wire; (b) a stiff tip; said tip is located at the proximal end of said guide-wire; or, (c) a bent tip; said tilt is located at the proximal end of said guide-wire; (d) a sharpened tip adapted for puncturetion of said occlusion and of the layers of said vessel; (e) is an elongated straight member or any combination thereof.

14. (canceled)

15. (canceled)

16. (canceled)

17. (canceled)

18. A method for crossing occlusions in vessels of a patient, comprising:
a. providing at least one catheter adapted to cross occlusions in vessels of a patient having a proximal end reversibly insertable within said occlusion and a distal opposite end located outside said patient, comprising:
i. at least one open-bore lumen provided in parallel to the main longitudinal axis of said catheter, having a proximal end located at said proximal end of said catheter, and a distal end located outside said patient;
ii. at least one vacuum generating mechanism (VGM), being in a fluid communication with said lumen's proximal end; said VGM is adapted for generating an effective measure of vacuum and introducing the vacuum towards said proximal end of said catheter, via a portion or an entire length of said lumen;
iii. at least one movable pusher; adapted to be reversibly reciprocate, by means of an actuating mechanism, along and around said main longitudinal axis of said catheter; said pusher is locatable between at least two configurations, a CLOSE configuration and an OPEN configuration, in said OPEN configuration, the proximal end of said pusher is intimately anchored within said occlusion; said pusher, in said OPEN configuration, provides within said vessel a preset void volume barriered by means of said pusher, said proximal end of said catheter and the inner surface of said occlusion;
iv. at least one guide-wire, accommodated within said lumen, reciprocally maneuvered along and around said main longitudinal axis of said lumen; said guide-wire is adapted for crossing said occlusion;
b. crossing said occlusion with said guide-wire or otherwise inserting said catheter to a location adjacent to said occlusion;
c. bringing said pusher into a said OPEN configuration by moving said pusher forward, or alternatively, by moving said catheter backward while keeping said pusher adjacent to said occlusion;
d. generating vacuum by said VGM and applying suction within said occlusion; thereby stabilizing said pusher and fixing the same to the inner surface of said occlusion and/or to said inner surface of said vessel so as to enable said guide-wire to cross said occlusion;
e. advancing said guide-wire through said lumen into said occlusion while said suction is applied and crossing said occlusion with said guide-wire;
f. advancing said catheter through said occlusion;
g.
h. moving said guide-wire to a direction distal to said occlusion; bringing pusher to said OPEN configuration; creating vacuum by said VGM and applying suction on said inner surface of said occlusion and/or on said inner surface of said vessel through said void volume; in this position, said pusher is stabilized and fixed to said inner surface of said occlusion and/or to said inner surface of said vessel; puncturating the intima of said vessel; advancing guide wire either straight or bent in the direction of the lumen in order to puncture the intima of said vessel in order to gain access to the lumen;
i. terminating the suction, once the guide-wire crossed said occlusion; and bringing the pusher into a said CLOSE configuration.

19. The method according to claim 18, further comprising steps of treating patient with CTO conditions.

20. The method according to claim 18, wherein said step of providing said catheter comprises at least one pushing wire; said pushing wire mechanically connected to said pusher.

21. (canceled)

22. (canceled)

23. The method according to claim 18, wherein said steps of providing said catheter and bringing said pusher into a said OPEN configuration, said lumen is selected from a group consisting of (a) a pusher-lumen; said pushing wire is accommodated within said pusher-lumen; (b) a suction lumen fluidly communicating with said VGM; (c) a guide-wire-lumen; said guide-wire is accommodated within said guide-wire-lumen; or any combination thereof.

24. (canceled)

25. (canceled)

26. The method according to claim 18, wherein said step of providing said catheter, said pusher is a solid cap having a tapered shape; said cap is advancing, penetrating, anchoring, piercing, crossing or otherwise actuating said catheter within said occlusion.

27. The method according to claim 18, wherein said step of providing said catheter, least a proximal portion of said pusher is a curled wire, characterized by a shape selected from a group consisting of helix, sphere, cone, cylinder, egg-like, and any combination thereof.

28. (canceled)

29. (canceled)

30. The method according to claim 18, wherein said step of providing said catheter, said guide-wire is characterized by at least one selected from a croup consisting of (a) a flexible tip; said tip is located at the proximal end of said guide-wire: or, (b) a stiff tip; said tip is located at the proximal end of said guide-wire; (c) an elongated straight member; (d) a bent tip; said tip is located at the proximal end of said guide-wire; (e) a sharpened tip adapted for puncturetion of said occlusion and of the layers of said vessel; or any combination.

31. (canceled)

32. (canceled)

33. (canceled)

34. (canceled)

35. The catheter according to claim 1, comprising:
a. at least one fluid pressure-pulse generator adapted to provide at least one fluid pulse with relatively steep potential difference between initiation and termination; and,
b. at least one lumen; said lumen is in communication with said fluid pressure-pulse generator;
wherein said pressure generator is adapted to provide said at least one fluid pulse through said catheter to said occlusion such that said occlusion is crossed.

36. The device according to claim 35, adapted to treat patients with CTO conditions.

37. (canceled)

38. The device according to claim 35, adapted to reverse the generation of pulses, and thereby to act in a suction manner.

39. A method for crossing an occlusion in vessels of a patient, comprising:
a. obtaining at least one catheter adapted to cross an occlusion in a hollow tube-like passage, comprising:
i. at least one fluid pressure-pulse generator adapted to provide at least one fluid pulse with relatively steep potential difference between initiation and termination; and,
ii. at least one lumen; said lumen is in communication with said fluid pressure-pulse generator;
b. attempting to cross the occlusion with a regular guide-wire;
c. bringing the tip of the hydraulic wire in contact with the occlusive lesion;
d. applying a primary series of hydraulic impacts; thereby enabling passage of a guide-wire through the said occlusion;
e. advancing the wire through the occlusion.

40. The method according to claim 39, additionally comprising at least one step selected from:
a. delivering a secondary series of hydraulic impacts to create localized disruption of the occluding tissue in order to enable passage of the wire;
b. entering the lumen beyond the occluded segment; and
c. verifying the position of the true lumen of said vessel and performing reentry with said guide-wire.
